(19) United States
(12) Patent Application Publication
(10) Pub. No.: US 2013/0142692 A1
(43) Pub. Date: Jun. 6, 2013

(54) METHODS AND APPARATUS FOR PURIFICATION OF AIR

(71) Applicant: Mohamed H. Tarifi, Derby, CT (US)

(72) Inventor: Mohamed H. Tarifi, Derby, CT (US)

(21) Appl. No.: 13/244,121

(22) Filed: Sep. 23, 2011

Related U.S. Application Data
(60) Provisional application No. 61/390,532, filed on Oct. 6, 2010.

(51) Int. Cl. A61L 9/20

(52) U.S. Cl. CPC A61L 9/205
USPC 422/4; 422/121

(57) ABSTRACT

Air is purified through the use of a photo catalytic oxidation (PCO) and ultraviolet C (UVC) photon treatment. A single-stage PCO/UVC reactor includes an activated carbon filter (ACF) having titanium dioxide embedded therewithin or thereupon. A UVC set of lamps are disposed upstream and downstream of the ACFs. The UVC sets of lamps impinge UVC photons on the ACFs, initiating the PCO process and completely oxidizing toxic VOCs into carbon dioxide and water. The ACFs act to absorb and hold the VOCs (and intermediate breakdown organic compounds, such as formaldehyde) until the PCO process can completely break down the VOCs into carbon dioxide and water. A multiple stage UVC/PCO reactor may be used, if required and depending on the concentration of contaminants in the incoming air. In addition to detoxification, the UVC and PCO together sterilize the air by destroying viruses, bacteria and molds.

### CROSS REFERENCE TO RELATED APPLICATIONS

This application claims the benefit of priority of U.S. provisional patent application No. 61/390,532, filed Oct. 6, 2010, the contents of which are herein incorporated by reference.

### BACKGROUND OF THE INVENTION

The present invention relates to methods and apparatus for the purification of air and, more particularly, to methods and apparatus for sterilizing air by photo catalytic oxidation (PCO) and ultraviolet C (UVC) irradiation, and breaking down toxic volatile organic compounds (VOCs) by oxidizing them to carbon dioxide and water.

The PCO process, given enough time, is capable of oxidizing toxic VOCs to carbon dioxide and water. However, simply flowing air through a PCO reactor may not provide sufficient reaction time, thereby producing toxic intermediate compounds, such as formaldehyde and acetyl aldehyde. Conventional systems often require chemical scrubbers downstream of the PCO reactor to remove these toxic organic compounds. Such systems are not cost effective and many such methods have been abandoned.

As can be seen, there is a need for improved methods and apparatus for removing toxic VOCs from air without requiring downstream chemical scrubbers or additional purification devices.

### SUMMARY OF THE INVENTION

In one aspect of the present invention, an air purification apparatus comprises a filter containing filter media, the filter media adapted to absorb pollutants from the air; titanium dioxide (TiO₂) immobilized on the filter media; a first ultraviolet C (UVC) lamp disposed on the upstream side of the filter; and a second UVC lamp disposed on the downstream side of the filter.

In another aspect of the present invention, an air purification apparatus comprises an activated carbon filter which absorbs pollutants from the air; titanium dioxide (TiO₂) immobilized on the activated carbon filter; a first ultraviolet C (UVC) lamp placed on the upstream side of the filter; a second UVC lamp placed on the downstream side of the filter; and a reflector covering the inside of the air duct to reflect UVC photons.

In a further aspect of the present invention, a method for the purification of an air stream comprises illuminating an activated carbon filter (ACF) with an ultraviolet C (UVC) lamp, wherein the ACF has TiO₂ immobilized thereupon; passing the air stream through the ACF; absorbing volatile organic compounds (VOCs) in the ACF; and oxidizing the absorbed VOCs by a photo catalytic oxidation (PCO) process.

UVC and PCO, working collaboratively, will eradicate any bacteria, virus, or mold present in the incoming air.

These and other features, aspects and advantages of the present invention will become better understood with reference to the following drawings, description and claims.

### BRIEF DESCRIPTION OF THE DRAWINGS

FIG. 1 is cross-sectional view of an apparatus for purifying air according to an exemplary embodiment of the present invention; and

FIG. 2 is a flow chart describing a method according to an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides methods and apparatus for the sterilization and detoxification of air through the use of a photo catalytic oxidation (PCO) and ultraviolet C (UVC) photons. A single stage PCO/UVC reactor includes one activated carbon filters (ACFs) having titanium dioxide embedded therewithin or thereupon. UVC lamp(s) may be disposed upstream and downstream of the ACFs. The UVC lamps impinge UVC photons on the ACFs, initiating the PCO process and oxidizing the VOCs into carbon dioxide and water. The ACFs act to absorb and hold the VOCs (and intermediate breakdown organic compounds, such as formaldehyde) until the PCO process can completely break down the VOCs into carbon dioxide and water. Multiple stage PCO/UVC may be used in series in an air duct. The air duct may be lined with a UVC reflector at and near the UVC lamps and the ACFs.

As used herewithin, the term “UVC lamp” or “UVC set of lamps” refers to one or more UVC germicidal lamps.

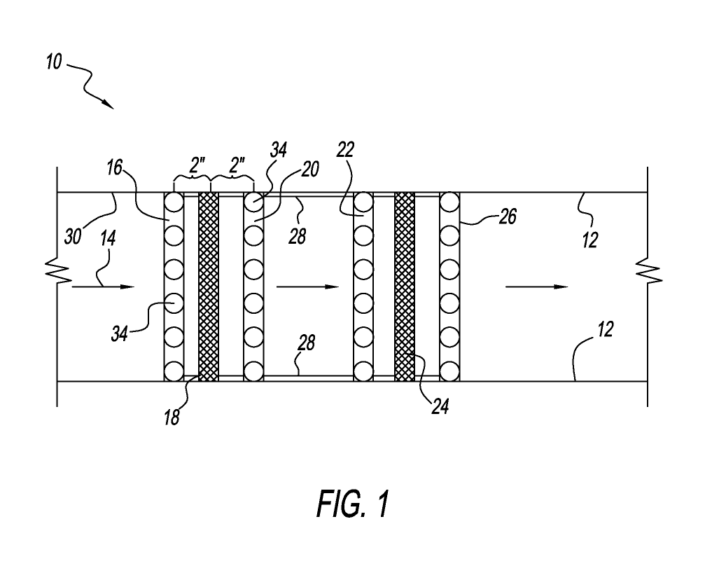

Referring to FIG. 1, a PCO/UVC reactor system 10 may be disposed in an air duct 12. The direction of the flow of air is indicated by arrow 14. A first UVC set of lamps 16 may be disposed upstream of a first ACF 18. A second UVC set of lamps 20 may be disposed downstream of the first ACF 18, thereby sandwiching the ACF 18 between the UVC lamp sets 16, 20. The UVC lamps 16, 20 may be spaced from about 0.5 inch to about 4 inches, typically about 2 inches, away from the first ACF 18. The UVC lamp sets 16, 20, combined with the first ACF 18, may form a one stage PCO/UVC reactor 10-1. A second PCO/UVC reactor 10-2 may be formed from a third UVC lamp 22, a second ACF 24 and a fourth UVC lamp 26 disposed downstream and in series with the first PCO/UVC reactor 10-1. PCO/UVC reactors 10-1 and 10-2 together form a two stage PCO/UVC reactor. Additional PCO/UVC stages (not shown) may be added depending on the volume and velocity of the flow of air and the concentration of the VOCs in the air stream.

A reflector 28 may be disposed on an inside surface 30 of the air duct 12. The reflector 28 may be disposed between the UV sets of lamps 16, 20 and the ACF 18. When multiple stage PCO/UVC reactor is used (such as PCO/UVC reactors 10-1 and 10-2), the reflector 28 may additionally line the inside surface 30 of the air duct 12 between the two reactors. The reflector 28 may be adapted to reflect UVC photon.

The UVC germicidal lamp sets 16, 20, 22, 26 may include one or more UVC lamps 34. For example, as shown in FIG. 1, six UVC lamps 34 may be disposed in the air duct 12. In some embodiments, the UVC lamps 34 may be disposed from one side to another side of the air duct 12 and the UVC lamps 34 may be equally spaced from one another to allow air flow 14 to pass through the UVC sets of lamps 16, 20, 22, 26. From one to 10 or more UVC lamps 34 may be disposed in the air duct 12 for each of the UVC sets of lamps 16, 20, 22, 26, depending on size of the UVC lamps 34 (both physical size and wattage), size of the air duct 12, and the like.

The ACFs 18, 24 may be any type of filter having filter media capable of absorbing VOCs. The ACFs 18, 24 can be a block, honey comb, refillable or any other suitable activated carbon media filter. All carbon media, along with any housing, if needed, may have TiO₂ immobilized thereupon. For example, TiO₂ may be immobilized on the activated carbon in the ACF 18, 24. If a fillable filter is used, a mesh screen housing 36 may be disposed to contain the loose activated carbon media. In this embodiment (fillable filter), TiO₂ may be immobilized on the mesh screen housing 36 in addition to the loose activated carbon particulates.

In some embodiments, the PCO/UVC reactor system 10 may be pre-formed as a separate unit that may fit inside of a standard sized duct. In other embodiments, the PCO/UVC reactor system 10 may be custom made to fit inside any duct size or shape. In still other embodiments, the PCO/UVC reactor system 10 may be configured as a unit designed to replace a section of duct.

While the ACFs 18, 24 described above are described as activated carbon filters, other filter media may be used as may be known in the art. Suitable filter media may include material capable of capturing and holding VOCs and releasing the carbon dioxide and water reactants when the PCO/UVC process breaks down the captured VOCs. The media may have a pore size adapted to capture and hold the desired VOCs to be removed from the air. In some embodiments, the media may have a range of pore sizes to be effective on a wide range of VOCs. The filter media may absorb the VOCs on its surface, allowing the PCO/UVC process ample time to complete the oxidation of VOCs to carbon dioxide and water.

While the above description describes using TiO₂ for the PCO process, other chemical combinations may be useful, such as binary oxides selected from TiO₂/SiO₂, TiO₂/ZrO₂, TiO₂/SnO₂, TiO₂/WO₃, TiO₂/MoO₃ and mixtures thereof.

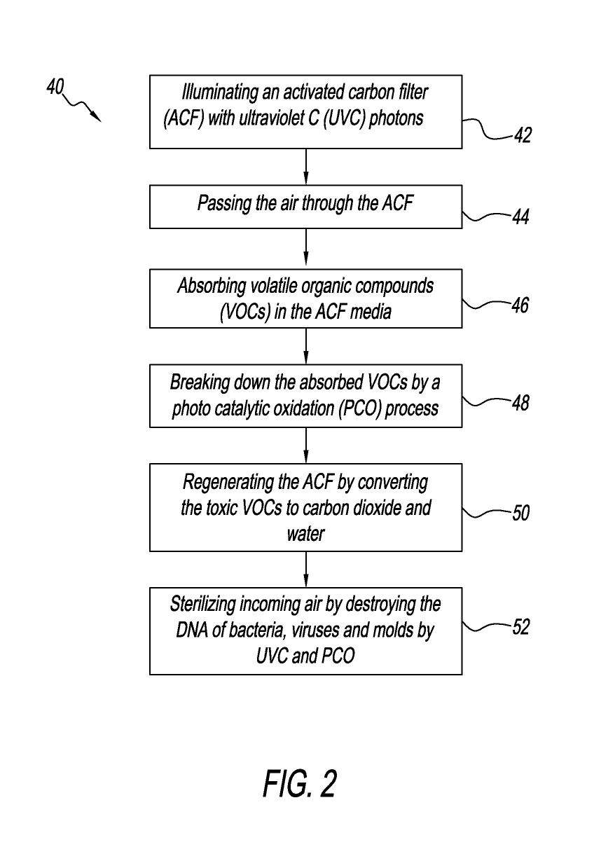

Referring now to FIG. 2, a method 40 for treating air includes a step 42 of illuminating an activated carbon filter (ACF) with ultraviolet C (UVC) photons, wherein the ACF has TiO2 immobilized there upon. The method 40 includes a further step 44 of passing the air through the ACF and a step 46 of absorbing volatile organic compounds (VOCs) in the ACF. The method 40 includes a step 48 of breaking down the absorbed VOCs by a photo catalytic oxidation (PCO) process. The method 40 may further include a step 50 of regenerating the ACF by releasing the decomposition products (carbon dioxide and water) of the VOCs. The method 40 may further include a step 52 of sterilizing incoming air by destroying the DNA of bacteria, viruses and molds by UVC and PCO.

The method and apparatus of the present invention may be used to not only completely oxidize VOCs to carbon dioxide and water, but also to destroy organisms in the air stream, allowing the present invention to not only detoxify the air stream, but to also sterilize the air stream. The method and apparatus of the present invention may be used in residential structures, commercial and industrial buildings, hospitals and confined spaces, such as submarines, airplanes, ships, and the like. The method and apparatus of the present invention may be adapted for use in a portable, self-standing room air purifier.

It should be understood, of course, that the foregoing relates to exemplary embodiments of the invention and that modifications may be made without departing from the spirit and scope of the invention as set forth in the following claims.

What is claimed is:

1. An air purification apparatus comprising:
a filter containing filter media, the filter media adapted to absorb pollutants from the air;
titanium dioxide (TiO₂) immobilized on the filter media;
a first ultraviolet C (UVC) set of lamps disposed on an upstream side of the filter; and
a second UVC set of lamps disposed on a downstream side of the filter, wherein the filter, first UVC set of lamps and the second UVC set of lamps together form a single stage UVC/PCO reactor.

2. The air purification apparatus of claim 1, wherein the filter is an activated carbon filter (ACF).

3. The air purification apparatus of claim 1, further comprising a reflector disposed to reflect UVC photons.

4. The air purification apparatus of claim 3, wherein the air purification apparatus is disposed in an air duct and the reflector is disposed on an inside surface of the air duct.

5. The air purification apparatus of claim 1, wherein the first and second UVC sets of lamps are disposed from about 0.5 inch to about 4 inches from the filter.

6. The air purification apparatus of claim 5, wherein the first and second UVC sets of lamps are disposed about 2 inches from the filter.

7. The air purification apparatus of claim 1, further comprising a third and fourth UVC set of lamps sandwiching a second filter, the third and fourth UVC sets of lamps and the second filter disposed comprise a second stage reactor in series with the first stage reactor comprising a two stage reactor.

8. The air purification apparatus of claim 1, further comprising a plurality of UVC lamps in each UVC set of lamps.

9. An air purification apparatus comprising:
an activated carbon filter adapted to absorb pollutants from the air;
titanium dioxide (TiO₂) immobilized on the activated carbon filter;
a first ultraviolet C (UVC) set of lamps disposed on an upstream side of the filter;
a second UVC set of lamps disposed on a downstream side of the filter; and
a reflector disposed to reflect UVC photons produced by the UVC sets of lamps.

10. The air purification apparatus of claim 9, wherein the air purification apparatus is disposed in an air duct and the reflector is disposed on an inside surface of the air duct.

11. The air purification apparatus of claim 9, wherein the first and second UVC sets of lamps are disposed from about 0.5 inch to about 4 inches from the filter.

12. The air purification apparatus of claim 11, wherein the first and second UVC sets of lamps are disposed about 2 inches from the filter.

13. The air purification apparatus of claim 9, further comprising a third and fourth UVC set of lamps sandwiching a second filter, the third and fourth UVC sets of lamps and the second filter disposed in series with the first and second UVC sets of lamps and the filter.

14. A method for the purification of an air stream, comprising:
illuminating an activated carbon filter (ACF) with ultraviolet C (UVC) photons, wherein the ACF has TiO₂ immobilized thereupon;
passing the air stream through the ACF;
absorbing volatile organic compounds (VOCs) in the ACF; and
oxidizing the absorbed VOCs by a photo catalytic oxidation (PCO) process.

15. The method of claim 14, further comprising regenerating the ACF by releasing complete oxidation by products of the VOCs.

16. The method of claim 15, wherein complete oxidation by products are carbon dioxide and water.

17. The method of claim 15, further comprising destroying organisms in the air stream.
